(19) United States
(12) Patent Application Publication
(10) Pub. No.: US 2025/0069723 A1
(43) Pub. Date: Feb. 27, 2025

(54) GENERATING TRAINING DATA FOR A NUTRITIONAL REPLACEMENT MACHINE-LEARNING MODEL

(71) Applicant: Maplebear Inc., San Francisco, CA (US)

(72) Inventors: Bhavya Gulati, Milipitas, CA (US); Chakshu Ahuja, San Jose, CA (US); Girija Narlikar, Palo Alto, CA (US); Karuna Ahuja, San Francisco, CA (US); Radhika Goel, San Francisco, CA (US)

(21) Appl. No.: 18/455,498

(22) Filed: Aug. 24, 2023

(51) Int. Cl. G16H 20/60; G16H 50/20

(52) U.S. Cl. CPC G16H 20/60; G16H 50/20

(57) ABSTRACT

The online concierge system accesses item data for a target item and item data for a candidate item. The online concierge system generates a replacement score based on the accessed item data and generates a nutrition score based on the item data for the candidate item. The online concierge system generates a nutrition replacement score based on the replacement score and the nutrition score and stores a training example based on the item data and the nutrition replacement score. The training example may include the item data for the target item and the candidate item and a label based on the nutrition replacement score.

### BACKGROUND

Online concierge systems allow customers to place online delivery orders and match the orders with pickers who service the orders at retailer locations on behalf of the customers. An online concierge system might recommend replacement items for a customer to consider as alternatives to items that a user is otherwise considering. For example, a user who is viewing content related to a bag of potato chips may be presented with content related to alternative items that the user may be interested in that are related to potato chips, such as tortilla chips or popcorn. However, items that are most relevant or similar to an item that a user is viewing will generally have a similar nutritional value to the target item. Thus, when a user is viewing an unhealthy item, online concierge systems that present similar items are likely to present similarly unhealthy items.

The task of balancing item relevance with nutrition becomes exacerbated when the online concierge system uses machine-learning models to select content to present to users. These machine-learning models are generally trained by automatically collecting training data describing instances where users have actually selected or rejected certain items as replacements for others. However, because this training data is collected based on the historical behavior of users, models trained based on this data tends to cause users to continue these behaviors. While this may be effective for predicting user behavior, it is ineffective at changing user behavior to select more nutritious items. Thus, traditional techniques for generating training data for machine-learning models are unable to properly train a machine-learning model to suggest nutritious alternatives that are also relevant to a target item.

### SUMMARY

In accordance with one or more aspects of the disclosure, to generate training examples for a nutritional replacement model, an online concierge system accesses item data for a target item and a candidate item. The candidate item is a candidate nutritional replacement for the target item. The online concierge system generates a replacement score for the candidate item that represents the likelihood that a user will select the candidate item as a replacement for the target item. For example, the online concierge system may apply a replacement model to predict this likelihood. In some embodiments, the online concierge system uses training examples for such a replacement model to generate training examples for the nutritional replacement model, and simply uses the replacement score labels from those training examples as replacement scores.

The online concierge system also generates nutrition scores for candidate items that represent the nutritional value of the candidate items. The online concierge system may automate the computation of these nutrition scores by identifying healthy recipes and computing the rates at which items appear in those healthy recipes versus non-healthy recipes. For example, the online concierge system may use natural language processing or large language models to identify recipes that are healthy based on the titles or descriptions of the recipes. The online concierge system may compute the rate at which items appear in healthy recipes and use that rate to compute a nutrition score for the item. The online concierge system may perform a similar process with the order history of users that the online concierge system identifies as “healthy” users, and may use the rate at which those users order items to compute nutrition scores for items.

The online concierge system uses the replacement scores and the nutrition scores to generate nutritional replacement scores. The online concierge system uses these nutritional replacement scores to generate training examples for a nutritional replacement model. More specifically, each training example for a nutritional replacement model may include item data for a target item and a candidate item, and a label that is based on the nutritional replacement score computed based on the target item and the candidate item.

The online concierge system trains the nutritional replacement model based on the generated training examples, and uses the nutritional replacement model to select nutritional replacements for items that users interact with. For example, the online concierge system may apply the nutritional replacement model for a set of candidate items as possible replacements for an item that the user has selected for an order or is viewing through a client application, and select a subset of the candidate items to present to the user through a client application.

By automating the generation of nutrition scores for items, the online concierge system can automate the generation of training examples for a nutritional replacement model. Thus, an online concierge system can efficiently and effectively train a machine-learning model that can change user behavior rather than simply predicting it.

### BRIEF DESCRIPTION OF THE DRAWINGS

FIG. 1 illustrates an example system environment for an online concierge system, in accordance with one or more embodiments.

FIG. 2 illustrates an example system architecture for an online concierge system, in accordance with one or more embodiments.

FIG. 3 illustrates an example data flow through modules of an online concierge system to generate training examples for a nutritional replacement model, in accordance with one or more embodiments.

FIG. 4 is a flowchart for a method of generating training examples for a nutritional replacement model, in accordance with one or more embodiments.

FIG. 5 illustrates an example user interface displaying nutritional replacements for a target item, in accordance with one or more embodiments.

### DETAILED DESCRIPTION

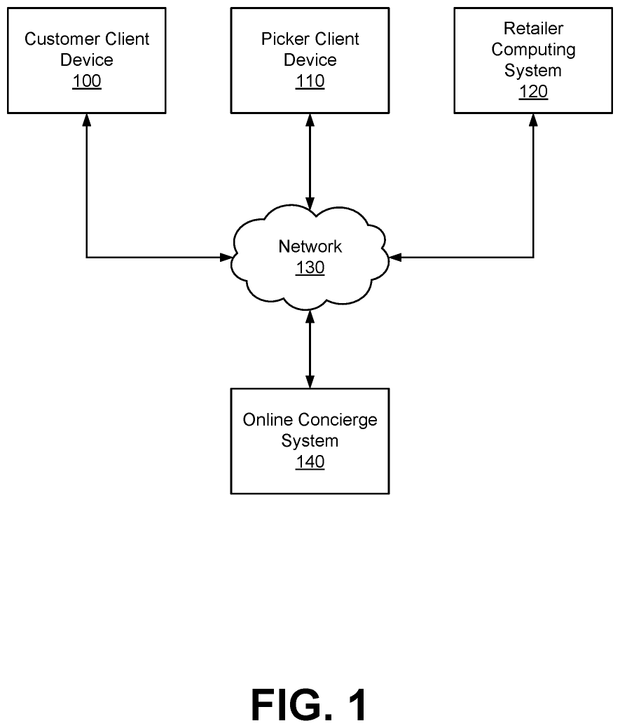

FIG. 1 illustrates an example system environment for an online concierge system 140, in accordance with one or more embodiments. The system environment illustrated in FIG. 1 includes a customer client device 100, a picker client device 110, a retailer computing system 120, a network 130, and an online concierge system 140. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

As used herein, customers, pickers, and retailers may be generically referred to as “users” of the online concierge system 140. Additionally, while one customer client device 100, picker client device 110, and retailer computing system 120 are illustrated in FIG. 1, any number of customers, pickers, and retailers may interact with the online concierge system 140. As such, there may be more than one customer client device 100, picker client device 110, or retailer computing system 120.

The customer client device 100 is a client device through which a customer may interact with the picker client device 110, the retailer computing system 120, or the online concierge system 140. The customer client device 100 can be a personal or mobile computing device, such as a smartphone, a tablet, a laptop computer, or desktop computer. In some embodiments, the customer client device 100 executes a client application that uses an application programming interface (API) to communicate with the online concierge system 140.

A customer uses the customer client device 100 to place an order with the online concierge system 140. An order specifies a set of items to be delivered to the customer. An “item”, as used herein, means a good or product that can be provided to the customer through the online concierge system 140. The order may include item identifiers (e.g., a stock keeping unit or a price look-up code) for items to be delivered to the user and may include quantities of the items to be delivered. Additionally, an order may further include a delivery location to which the ordered items are to be delivered and a timeframe during which the items should be delivered. In some embodiments, the order also specifies one or more retailers from which the ordered items should be collected.

The customer client device 100 presents an ordering interface to the customer. The ordering interface is a user interface that the customer can use to place an order with the online concierge system 140. The ordering interface may be part of a client application operating on the customer client device 100. The ordering interface allows the customer to search for items that are available through the online concierge system 140 and the customer can select which items to add to a “shopping list.” A “shopping list,” as used herein, is a tentative set of items that the user has selected for an order but that has not yet been finalized for an order. The ordering interface allows a customer to update the shopping list, e.g., by changing the quantity of items, adding or removing items, or adding instructions for items that specify how the item should be collected.

The customer client device 100 may receive additional content from the online concierge system 140 to present to a customer. For example, the customer client device 100 may receive coupons, recipes, or item suggestions. The customer client device 100 may present the received additional content to the customer as the customer uses the customer client device 100 to place an order (e.g., as part of the ordering interface).

Additionally, the customer client device 100 includes a communication interface that allows the customer to communicate with a picker that is servicing the customer's order. This communication interface allows the user to input a text-based message to transmit to the picker client device 110 via the network 130. The picker client device 110 receives the message from the customer client device 100 and presents the message to the picker. The picker client device 110 also includes a communication interface that allows the picker to communicate with the customer. The picker client device 110 transmits a message provided by the picker to the customer client device 100 via the network 130. In some embodiments, messages sent between the customer client device 100 and the picker client device 110 are transmitted through the online concierge system 140. In addition to text messages, the communication interfaces of the customer client device 100 and the picker client device 110 may allow the customer and the picker to communicate through audio or video communications, such as a phone call, a voice-over-IP call, or a video call.

The picker client device 110 is a client device through which a picker may interact with the customer client device 100, the retailer computing system 120, or the online concierge system 140. The picker client device 110 can be a personal or mobile computing device, such as a smartphone, a tablet, a laptop computer, or desktop computer. In some embodiments, the picker client device 110 executes a client application that uses an application programming interface (API) to communicate with the online concierge system 140.

The picker client device 110 receives orders from the online concierge system 140 for the picker to service. A picker services an order by collecting the items listed in the order from a retailer. The picker client device 110 presents the items that are included in the customer's order to the picker in a collection interface. The collection interface is a user interface that provides information to the picker on which items to collect for a customer's order and the quantities of the items. In some embodiments, the collection interface provides multiple orders from multiple customers for the picker to service at the same time from the same retailer location. The collection interface further presents instructions that the customer may have included related to the collection of items in the order. Additionally, the collection interface may present a location of each item at the retailer, and may even specify a sequence in which the picker should collect the items for improved efficiency in collecting items. In some embodiments, the picker client device 110 transmits to the online concierge system 140 or the customer client device 100 which items the picker has collected in real time as the picker collects the items.

The picker can use the picker client device 110 to keep track of the items that the picker has collected to ensure that the picker collects all of the items for an order. The picker client device 110 may include a barcode scanner that can determine an item identifier encoded in a barcode coupled to an item. The picker client device 110 compares this item identifier to items in the order that the picker is servicing, and if the item identifier corresponds to an item in the order, the picker client device 110 identifies the item as collected. In some embodiments, rather than or in addition to using a barcode scanner, the picker client device 110 captures one or more images of the item and determines the item identifier for the item based on the images. The picker client device 110 may determine the item identifier directly or by transmitting the images to the online concierge system 140. Furthermore, the picker client device 110 determines a weight for items that are priced by weight. The picker client device 110 may prompt the picker to manually input the weight of an item or may communicate with a weighing system in the retailer location to receive the weight of an item.

When the picker has collected all of the items for an order, the picker client device 110 instructs a picker on where to deliver the items for a customer's order. For example, the picker client device 110 displays a delivery location from the order to the picker. The picker client device 110 also provides navigation instructions for the picker to travel from the retailer location to the delivery location. Where a picker is servicing more than one order, the picker client device 110 identifies which items should be delivered to which delivery location. The picker client device 110 may provide navigation instructions from the retailer location to each of the delivery locations. The picker client device 110 may receive one or more delivery locations from the online concierge system 140 and may provide the delivery locations to the picker so that the picker can deliver the corresponding one or more orders to those locations. The picker client device 110 may also provide navigation instructions for the picker from the retailer location from which the picker collected the items to the one or more delivery locations.

In some embodiments, the picker client device 110 tracks the location of the picker as the picker delivers orders to delivery locations. The picker client device 110 collects location data and transmits the location data to the online concierge system 140. The online concierge system 140 may transmit the location data to the customer client device 100 for display to the customer such that the customer can keep track of when their order will be delivered. Additionally, the online concierge system 140 may generate updated navigation instructions for the picker based on the picker's location. For example, if the picker takes a wrong turn while traveling to a delivery location, the online concierge system 140 determines the picker's updated location based on location data from the picker client device 110 and generates updated navigation instructions for the picker based on the updated location.

In one or more embodiments, the picker is a single person who collects items for an order from a retailer location and delivers the order to the delivery location for the order. Alternatively, more than one person may serve the role as a picker for an order. For example, multiple people may collect the items at the retailer location for a single order. Similarly, the person who delivers an order to its delivery location may be different from the person or people who collected the items from the retailer location. In these embodiments, each person may have a picker client device 110 that they can use to interact with the online concierge system 140.

Additionally, while the description herein may primarily refer to pickers as humans, in some embodiments, some or all of the steps taken by the picker may be automated. For example, a semi- or fully-autonomous robot may collect items in a retailer location for an order and an autonomous vehicle may deliver an order to a customer from a retailer location.

The retailer computing system 120 is a computing system operated by a retailer that interacts with the online concierge system 140. As used herein, a “retailer” is an entity that operates a “retailer location,” which is a store, warehouse, or other building from which a picker can collect items. The retailer computing system 120 stores and provides item data to the online concierge system 140 and may regularly update the online concierge system 140 with updated item data. For example, the retailer computing system 120 provides item data indicating which items are available at a retailer location and the quantities of those items. Additionally, the retailer computing system 120 may transmit updated item data to the online concierge system 140 when an item is no longer available at the retailer location. Additionally, the retailer computing system 120 may provide the online concierge system 140 with updated item prices, sales, or availabilities. Additionally, the retailer computing system 120 may receive payment information from the online concierge system 140 for orders serviced by the online concierge system 140. Alternatively, the retailer computing system 120 may provide payment to the online concierge system 140 for some portion of the overall cost of a user's order (e.g., as a commission).

The customer client device 100, the picker client device 110, the retailer computing system 120, and the online concierge system 140 can communicate with each other via the network 130. The network 130 is a collection of computing devices that communicate via wired or wireless connections. The network 130 may include one or more local area networks (LANs) or one or more wide area networks (WANs). The network 130, as referred to herein, is an inclusive term that may refer to any or all of standard layers used to describe a physical or virtual network, such as the physical layer, the data link layer, the network layer, the transport layer, the session layer, the presentation layer, and the application layer. The network 130 may include physical media for communicating data from one computing device to another computing device, such as MPLS lines, fiber optic cables, cellular connections (e.g., 3G, 4G, or 5G spectra), or satellites. The network 130 also may use networking protocols, such as TCP/IP, HTTP, SSH, SMS, or FTP, to transmit data between computing devices. In some embodiments, the network 130 may include Bluetooth or near-field communication (NFC) technologies or protocols for local communications between computing devices. The network 130 may transmit encrypted or unencrypted data.

The online concierge system 140 is an online system by which customers can order items to be provided to them by a picker from a retailer. The online concierge system 140 receives orders from a customer client device 100 through the network 130. The online concierge system 140 selects a picker to service the customer's order and transmits the order to a picker client device 110 associated with the picker. The picker collects the ordered items from a retailer location and delivers the ordered items to the customer. The online concierge system 140 may charge a customer for the order and provides portions of the payment from the customer to the picker and the retailer.

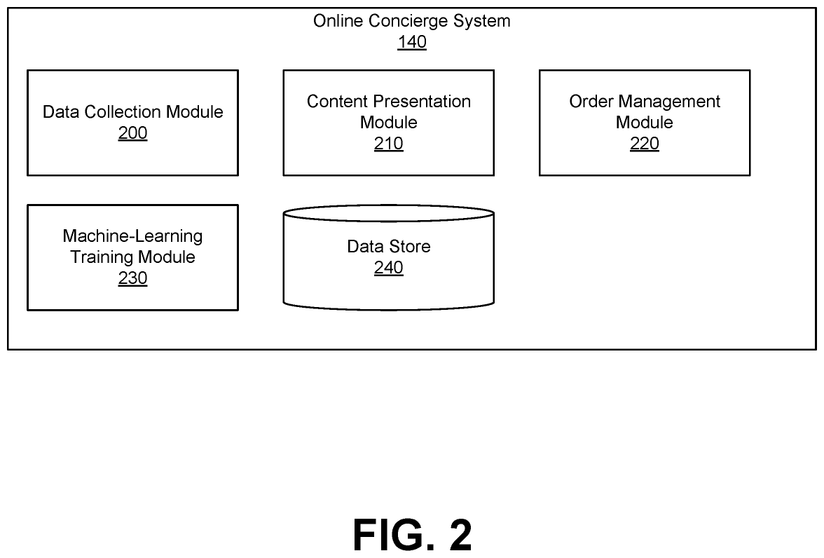

As an example, the online concierge system 140 may allow a customer to order groceries from a grocery store retailer. The customer's order may specify which groceries they want delivered from the grocery store and the quantities of each of the groceries. The customer's client device 100 transmits the customer's order to the online concierge system 140 and the online concierge system 140 selects a picker to travel to the grocery store retailer location to collect the groceries ordered by the customer. Once the picker has collected the groceries ordered by the customer, the picker delivers the groceries to a location transmitted to the picker client device 110 by the online concierge system 140. The online concierge system 140 is described in further detail below with regards to FIG. 2.

FIG. 2 illustrates an example system architecture for an online concierge system 140, in accordance with some embodiments. The system architecture illustrated in FIG. 2 includes a data collection module 200, a content presentation module 210, an order management module 220, a machine learning training module 230, and a data store 240. Alternative embodiments may include more, fewer, or different components from those illustrated in FIG. 2, and the functionality of each component may be divided between the components differently from the description below. Additionally, each component may perform their respective functionalities in response to a request from a human, or automatically without human intervention.

The data collection module 200 collects data used by the online concierge system 140 and stores the data in the data store 240. The data collection module 200 may only collect data describing a user if the user has previously explicitly consented to the online concierge system 140 collecting data describing the user. Additionally, the data collection module 200 may encrypt all data, including sensitive or personal data, describing users.

For example, the data collection module 200 collects customer data, which is information or data that describe characteristics of a customer. Customer data may include a customer's name, address, shopping preferences, favorite items, or stored payment instruments. The customer data also may include default settings established by the customer, such as a default retailer/retailer location, payment instrument, delivery location, or delivery timeframe. The data collection module 200 may collect the customer data from sensors on the customer client device 100 or based on the customer's interactions with the online concierge system 140.

The data collection module 200 also collects item data, which is information or data that identifies and describes items that are available at a retailer location. The item data may include item identifiers for items that are available and may include quantities of items associated with each item identifier. Additionally, item data may also include attributes of items such as the size, color, weight, stock keeping unit (SKU), or serial number for the item. The item data may further include purchasing rules associated with each item, if they exist. For example, age-restricted items such as alcohol and tobacco are flagged accordingly in the item data. Item data may also include information that is useful for predicting the availability of items in retailer locations. For example, for each item-retailer combination (a particular item at a particular warehouse), the item data may include a time that the item was last found, a time that the item was last not found (a picker looked for the item but could not find it), the rate at which the item is found, or the popularity of the item. The data collection module 200 may collect item data from a retailer computing system 120, a picker client device 110, or the customer client device 100.

An item category is a set of items that are a similar type of item. Items in an item category may be considered to be equivalent to each other or that may be replacements for each other in an order. For example, different brands of sourdough bread may be different items, but these items may be in a “sourdough bread” item category. The item categories may be human-generated and human-populated with items. The item categories also may be generated automatically by the online concierge system 140 (e.g., using a clustering algorithm).

The data collection module 200 also collects picker data, which is information or data that describes characteristics of pickers. For example, the picker data for a picker may include the picker's name, the picker's location, how often the picker has services orders for the online concierge system 140, a customer rating for the picker, which retailers the picker has collected items at, or the picker's previous shopping history. Additionally, the picker data may include preferences expressed by the picker, such as their preferred retailers to collect items at, how far they are willing to travel to deliver items to a customer, how many items they are willing to collect at a time, timeframes within which the picker is willing to service orders, or payment information by which the picker is to be paid for servicing orders (e.g., a bank account). The data collection module 200 collects picker data from sensors of the picker client device 110 or from the picker's interactions with the online concierge system 140.

Additionally, the data collection module 200 collects order data, which is information or data that describes characteristics of an order. For example, order data may include item data for items that are included in the order, a delivery location for the order, a customer associated with the order, a retailer location from which the customer wants the ordered items collected, or a timeframe within which the customer wants the order delivered. Order data may further include information describing how the order was serviced, such as which picker serviced the order, when the order was delivered, or a rating that the customer gave the delivery of the order. In some embodiments, the order data includes user data for users associated with the order, such as customer data for a customer who placed the order or picker data for a picker who serviced the order.

The content presentation module 210 selects content for presentation to a customer. For example, the content presentation module 210 selects which items to present to a customer while the customer is placing an order. The content presentation module 210 generates and transmits the ordering interface for the customer to order items. The content presentation module 210 populates the ordering interface with items that the customer may select for adding to their order. In some embodiments, the content presentation module 210 presents a catalog of all items that are available to the customer, which the customer can browse to select items to order. The content presentation module 210 also may identify items that the customer is most likely to order and present those items to the customer. For example, the content presentation module 210 may score items and rank the items based on their scores. The content presentation module 210 displays the items with scores that exceed some threshold (e.g., the top n items or the p percentile of items).

The content presentation module 210 may use an item selection model to score items for presentation to a customer. An item selection model is a machine learning model that is trained to score items for a customer based on item data for the items and customer data for the customer. For example, the item selection model may be trained to determine a likelihood that the customer will order the item. In some embodiments, the item selection model uses item embeddings describing items and customer embeddings describing customers to score items. These item embeddings and customer embeddings may be generated by separate machine learning models and may be stored in the data store 240.

In some embodiments, the content presentation module 210 scores items based on a search query received from the customer client device 100. A search query is free text for a word or set of words that indicate items of interest to the customer. The content presentation module 210 scores items based on a relatedness of the items to the search query. For example, the content presentation module 210 may apply natural language processing (NLP) techniques to the text in the search query to generate a search query representation (e.g., an embedding) that represents characteristics of the search query. The content presentation module 210 may use the search query representation to score candidate items for presentation to a customer (e.g., by comparing a search query embedding to an item embedding).

In some embodiments, the content presentation module 210 scores items based on a predicted availability of an item. The content presentation module 210 may use an availability model to predict the availability of an item. An availability model is a machine learning model that is trained to predict the availability of an item at a retailer location. For example, the availability model may be trained to predict a likelihood that an item is available at a retailer location or may predict an estimated number of items that are available at a retailer location. The content presentation module 210 may weight the score for an item based on the predicted availability of the item. Alternatively, the content presentation module 210 may filter out items from presentation to a customer based on whether the predicted availability of the item exceeds a threshold.

The order management module 220 that manages orders for items from customers. The order management module 220 receives orders from a customer client device 100 and assigns the orders to pickers for service based on picker data. For example, the order management module 220 assigns an order to a picker based on the picker's location and the location of the retailer from which the ordered items are to be collected. The order management module 220 may also assign an order to a picker based on how many items are in the order, a vehicle operated by the picker, the delivery location, the picker's preferences on how far to travel to deliver an order, the picker's ratings by customers, or how often a picker agrees to service an order.

In some embodiments, the order management module 220 determines when to assign an order to a picker based on a delivery timeframe requested by the customer with the order. The order management module 220 computes an estimated amount of time that it would take for a picker to collect the items for an order and deliver the ordered item to the delivery location for the order. The order management module 220 assigns the order to a picker at a time such that, if the picker immediately services the order, the picker is likely to deliver the order at a time within the timeframe. Thus, when the order management module 220 receives an order, the order management module 220 may delay in assigning the order to a picker if the timeframe is far enough in the future.

When the order management module 220 assigns an order to a picker, the order management module 220 transmits the order to the picker client device 110 associated with the picker. The order management module 220 may also transmit navigation instructions from the picker's current location to the retailer location associated with the order. If the order includes items to collect from multiple retailer locations, the order management module 220 identifies the retailer locations to the picker and may also specify a sequence in which the picker should visit the retailer locations.

The order management module 220 may track the location of the picker through the picker client device 110 to determine when the picker arrives at the retailer location. When the picker arrives at the retailer location, the order management module 220 transmits the order to the picker client device 110 for display to the picker. As the picker uses the picker client device 110 to collect items at the retailer location, the order management module 220 receives item identifiers for items that the picker has collected for the order. In some embodiments, the order management module 220 receives images of items from the picker client device 110 and applies computer-vision techniques to the images to identify the items depicted by the images. The order management module 220 may track the progress of the picker as the picker collects items for an order and may transmit progress updates to the customer client device 100 that describe which items have been collected for the customer's order.

In some embodiments, the order management module 220 tracks the location of the picker within the retailer location. The order management module 220 uses sensor data from the picker client device 110 or from sensors in the retailer location to determine the location of the picker in the retailer location. The order management module 220 may transmit to the picker client device 110 instructions to display a map of the retailer location indicating where in the retailer location the picker is located. Additionally, the order management module 220 may instruct the picker client device 110 to display the locations of items for the picker to collect, and may further display navigation instructions for how the picker can travel from their current location to the location of a next item to collect for an order.

The order management module 220 determines when the picker has collected all of the items for an order. For example, the order management module 220 may receive a message from the picker client device 110 indicating that all of the items for an order have been collected. Alternatively, the order management module 220 may receive item identifiers for items collected by the picker and determine when all of the items in an order have been collected. When the order management module 220 determines that the picker has completed an order, the order management module 220 transmits the delivery location for the order to the picker client device 110. The order management module 220 may also transmit navigation instructions to the picker client device 110 that specify how to travel from the retailer location to the delivery location, or to a subsequent retailer location for further item collection. The order management module 220 tracks the location of the picker as the picker travels to the delivery location for an order, and updates the customer with the location of the picker so that the customer can track the progress of their order. In some embodiments, the order management module 220 computes an estimated time of arrival for the picker at the delivery location and provides the estimated time of arrival to the customer.

In some embodiments, the order management module 220 facilitates communication between the customer client device 100 and the picker client device 110. As noted above, a customer may use a customer client device 100 to send a message to the picker client device 110. The order management module 220 receives the message from the customer client device 100 and transmits the message to the picker client device 110 for presentation to the picker. The picker may use the picker client device 110 to send a message to the customer client device 100 in a similar manner.

The order management module 220 coordinates payment by the customer for the order. The order management module 220 uses payment information provided by the customer (e.g., a credit card number or a bank account) to receive payment for the order. In some embodiments, the order management module 220 stores the payment information for use in subsequent orders by the customer. The order management module 220 computes a total cost for the order and charges the customer that cost. The order management module 220 may provide a portion of the total cost to the picker for servicing the order, and another portion of the total cost to the retailer.

The machine learning training module 230 trains machine learning models used by the online concierge system 140. The online concierge system 140 may use machine learning models to perform functionalities described herein. Example machine learning models include regression models, support vector machines, naïve bayes, decision trees, k nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers.

Each machine learning model includes a set of parameters. A set of parameters for a machine learning model are parameters that the machine learning model uses to process an input. For example, a set of parameters for a linear regression model may include weights that are applied to each input variable in the linear combination that comprises the linear regression model. Similarly, the set of parameters for a neural network may include weights and biases that are applied at each neuron in the neural network. The machine learning training module 230 generates the set of parameters for a machine learning model by “training” the machine learning model. Once trained, the machine learning model uses the set of parameters to transform inputs into outputs.

The machine learning training module 230 trains a machine learning model based on a set of training examples. Each training example includes input data to which the machine learning model is applied to generate an output. For example, each training example may include customer data, picker data, item data, or order data. In some cases, the training examples also include a label which represents an expected output of the machine learning model. In these cases, the machine learning model is trained by comparing its output from input data of a training example to the label for the training example.

The machine learning training module 230 may apply an iterative process to train a machine learning model whereby the machine learning training module 230 trains the machine learning model on each of the set of training examples. To train a machine learning model based on a training example, the machine learning training module 230 applies the machine learning model to the input data in the training example to generate an output. The machine learning training module 230 scores the output from the machine learning model using a loss function. A loss function is a function that generates a score for the output of the machine learning model such that the score is higher when the machine learning model performs poorly and lower when the machine learning model performs well. In cases where the training example includes a label, the loss function is also based on the label for the training example. Some example loss functions include the mean square error function, the mean absolute error, hinge loss function, and the cross entropy loss function. The machine learning training module 230 updates the set of parameters for the machine learning model based on the score generated by the loss function.

For example, the machine learning training module 230 may apply gradient descent to update the set of parameters.

The data store 240 stores data used by the online concierge system 140. For example, the data store 240 stores customer data, item data, order data, and picker data for use by the online concierge system 140. The data store 240 also stores trained machine learning models trained by the machine learning training module 230. For example, the data store 240 may store the set of parameters for a trained machine learning model on one or more non-transitory, computer-readable media. The data store 240 uses computer-readable media to store data, and may use databases to organize the stored data.

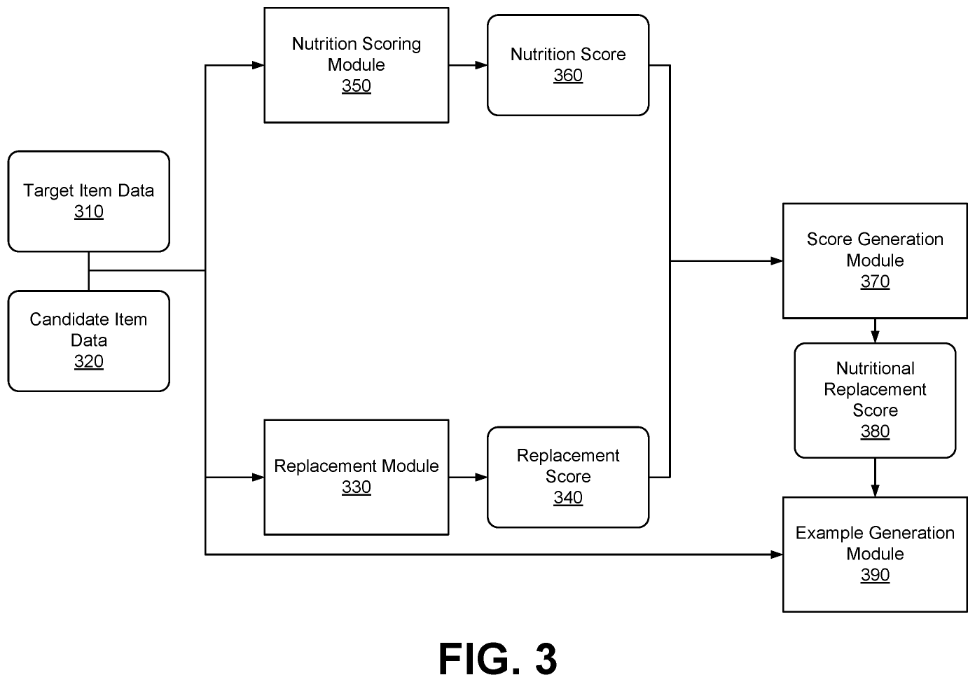

FIG. 3 illustrates an example data flow through modules of an online concierge system to generate training examples for a nutritional replacement model, in accordance with some embodiments. Alternative embodiments may include more, fewer, or different steps from those illustrated in FIG. 3, and the steps may be performed in a different order from that illustrated in FIG. 3. These steps may be performed by an online concierge system (e.g., online concierge system 140). Additionally, each of these steps may be performed automatically by the online concierge system without human intervention.

The online concierge system 140 accesses item data 310 for a target item and item data 320 for a candidate item. A candidate item is an item that a user may be interested in selecting as a replacement for the target item. For example, if the user is viewing a page related to the target item, the candidate item may be a candidate for display next to the target item as a possible replacement for the target item. Item data for the target item and the candidate item describes characteristics of each item. For example, the item data may describe an associated cost of the item, the user's history of ordering the item, and recipes that include the item. Item data may additionally include nutritional information including nutrient content such as vitamins, protein, fat, fiber, sodium, and calories, as well as other characteristic information.

The target item and the candidate item are items to be used for a training example for a nutritional replacement model. To select a target item and a candidate item, the online concierge system may randomly select a target item and a candidate item from a catalogue of items for the online concierge system. Similarly, the online concierge system may use target items and candidate items from training examples used to train a replacement model for the online concierge system.

The replacement module 330 computes a replacement score 340 for a candidate item and target item. The replacement score 340 represents the likelihood that the candidate item will be selected by a user as a replacement for the target item. In embodiments where the candidate item and the target item were items from a training example used to train a replacement model for the online concierge system, the replacement module 330 may simply use a label assigned to the corresponding training example as the replacement score 340. Alternatively, the replacement module 330 may compute a replacement score based on the item data of the items. For example, The replacement module 330 may use factors in the item data including a taste profile for each item, common recipes that use either item, prices, and nutritional information to determine whether the user is likely to approve the candidate item.

In some embodiments, the replacement module 330 uses a replacement model to generate a replacement score for a target item and a candidate item. A replacement model is a machine-learning model that is trained to generate a replacement score representing the likelihood that a user would replace the target item with the target item. As noted above, the replacement model may be generated based on a set of training examples. Each training example may represent a user's action after the online concierge system presented a candidate item as a replacement for a target item. For example, each training example may include item data for a target item, item data for a candidate item, and a label that indicates whether a user selected the candidate item as a replacement for the target item. In some embodiments, these training examples represent instances where the online concierge system notified the user that the target item was unavailable for an order and asked the user whether the candidate item was an acceptable replacement for the target item. The replacement model may weigh information in the target item data 310 and candidate item data 320 more or less heavily based on user data. User data may include the user's order history and dietary restrictions. User data may further include customer data. Customer data may include a customer's name, address, shopping preferences, favorite items, or stored payment instruments. The customer data also may include default settings established by the customer, such as a default retailer/retailer location, payment instrument, delivery location, or delivery timeframe. For example, when determining a replacement score 340 for a user, the replacement module may determine that the user has never approved a replacement item with a higher gluten content than a target item. The replacement model may therefore weigh candidate items with a lower gluten content or that are gluten free heavier than candidate replacement items with a higher gluten content than the target item. In this way, the replacement model may generate higher scores for candidate items that are determined to be similar to the target items, while accounting for the user's historical preferences.

The nutrition scoring module 350 generates a nutrition score 360 based on the item data for the target item and the candidate item. A nutrition score is a score that represents a nutritional value of a candidate item. A higher nutrition score 360 may indicate a healthier candidate item and a lower nutrition score 360 may indicate a less healthy candidate item. In some embodiments, the nutrition score represents the nutritional value of a candidate item. The nutrition score may correspond to nutritional metrics. Example nutritional metrics include nutrition content, such as fat, vitamins, fiber, sodium, and calories. The nutrition score also may be a combination of these metrics. In some embodiments, the nutrition score is absolute, such that the nutrition score is set based on the item's metrics. In some embodiments, the nutrition score is relative to the target item. For example, a bran muffin may have a nutrition score that is higher relative to chocolate and coffee cake muffins.

The nutrition scoring module 350 may compute a nutrition score 360 for a candidate item

based on item data for the candidate item. Item data for an item may include nutritional information describing nutritional values of the item (e.g., calories, sodium content, fat content, carb content, vitamin content). The online concierge system may compute a nutrition score for an item based on a function of these nutritional values for the item, such as a linear combination.

In some embodiments, the nutrition scoring module 350 uses a nutrition scoring model to compute a nutrition score for an item. A nutrition scoring model is a machine-learning model that is trained to compute a nutrition score for an item based on item data. The nutrition scoring model is trained based on a set of training examples. Each training example includes item data for an item and a label for the nutrition score that the machine-learning model should generate based on the item data. In embodiments where a nutrition score represents a relative nutritional value of a candidate item to a target item, the training examples may include item data for a candidate item and a target item.

In some embodiments, the labels for the training examples may be created manually using the input of one or more nutritionists. For example, one or more nutritionists may be provided item data for a set of items and may be asked to provide a nutrition score for each item, indicating the nutritional value of the item. The label for each training example may be based on the nutrition scores provided by multiple nutritionists (e.g., the average). These labels may then be used to train the nutrition scoring model.

In some embodiments, the nutrition scoring module 350 identifies certain users as healthy users and labels training examples with nutrition score labels based on whether those users order those items. The nutrition scoring module 350 may infer from a user's order history that the user tends to order healthy items. The nutrition scoring module 350 may then compute a nutrition score label for an item based on the rate at which these healthy users order the item.

In some embodiments, the nutrition scoring module 350 determines the nutrition score by comparing items in “healthy” recipes with items with recipes that are not “healthy.” The nutrition scoring module 350 may identify healthy recipes as recipes that have certain key words in their titles or descriptions, such as “low carb,” “low fat,” “healthy,” or “plant based.” In some embodiments, the nutrition scoring module 350 applies natural language processing techniques or a large-language model to the title or description of the recipe to identify whether a recipe is “healthy.” The nutrition scoring module 350 can use these classifications of “healthy” and not “healthy” recipes to compute nutrition scores for items. For example, the nutrition scoring module 350 may compute a nutrition score for an item based on the rates at which the item appears in healthy vs. not healthy recipes. Similarly, the nutrition scoring module 350 may compare similar healthy vs. not healthy recipes and use differences in the items used to compute nutrition scores. For example, if a “healthy” soup recipe uses low sodium vegetable broth whereas a not “healthy” recipe uses beef broth, the nutrition scoring module 350 may assign low sodium vegetable broth a higher nutrition score relative to beef broth.

The online concierge system 140 generates a nutritional replacement score 380 using a score generation module 370. The online concierge system 140 provides the score generation module 370 with the nutrition score 360 and the replacement score 340 for the target and candidate item pair. The nutritional replacement score 380 combines the two scores to reflect both a healthiness and a replacement viability of the candidate item for the target item. The nutritional replacement score 380 accounts for the candidate item as a replacement for the target item while also accounting for the nutritional value of the candidate item. The score generation module 370 may compute the nutritional replacement score 380 based on a function of the nutrition score 360 and replacement score 340 computed for the candidate item and the target item. For example, the score generation module 370 may use a linear combination or a product of the nutrition score 360 and the replacement score 340 to compute the nutritional replacement score.

The online concierge system 140 uses the example generation module 390 to store a training example for a nutritional replacement model. Each training example includes the item data 310 from the target item, the item data 320 from the candidate item, and the nutritional replacement score 380 as a label for the training example. These training examples can be used to train a nutritional replacement model that computes nutritional replacement scores based on a target item's item data and a candidate item's item data. In embodiments where replacement scores are generated based on user data, the training examples for a nutritional replacement model may include the user data used to compute the replacement scores to thereby train the nutritional replacement model to generate nutritional replacement scores based on the user to whom replacements may be suggested.

The online concierge system may use the trained nutritional replacement model to provide nutritional replacement options to users through a client application. For example, the online concierge system may identify a set of candidate items for a target item that a user is interacting with (e.g., viewing in a client application or added to the user's order). The online concierge system may use the nutritional replacement model to generate nutritional replacement scores for the candidate items and select one or more candidate items to present to the user through the user interface.

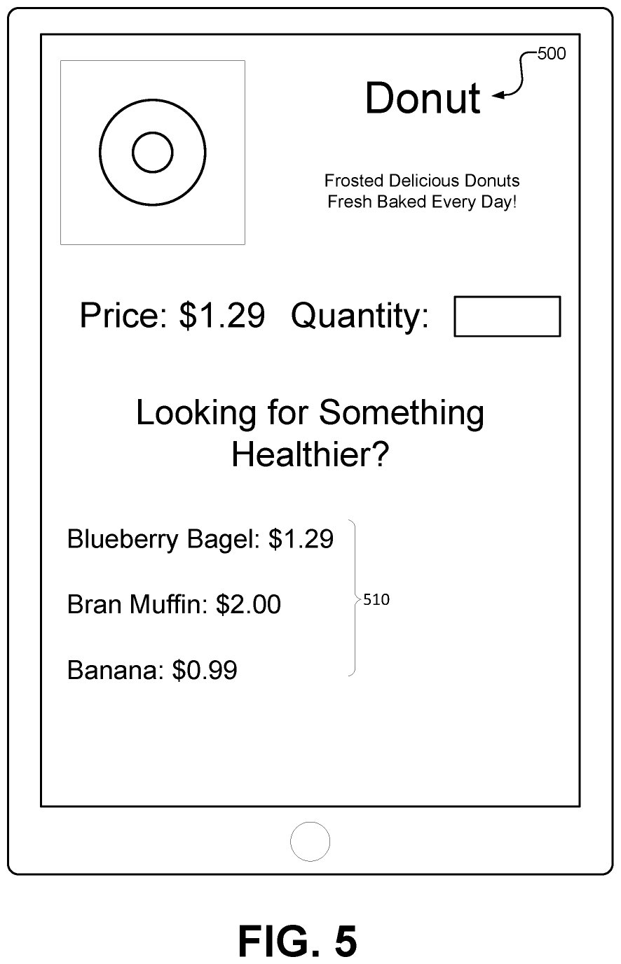

FIG. 5 illustrates an example user interface displaying nutritional replacements for a target item, in accordance with some embodiments. In FIG. 5, the online concierge system displays a page for the target item 500, which is a donut. The online concierge system presents certain candidate items 510 along with the target item based on how relevant those candidate items are to the target item 500 and how nutritious they are. For example, a banana may be a less relevant replacement to a donut, but may be much more nutritious. Similarly, while a blueberry bagel may not be quite as nutritious as a banana, it may be more relevant to the donut.

The nutritional replacement model may be iteratively trained based on updated nutritional replacement scores 380. As the online concierge system 140 generates new nutritional replacement scores 380 for target and candidate item pairs, the nutritional replacement model may be retrained. In some embodiments, the nutritional replacement model is retrained in response to the addition of new items to the online concierge system 140.

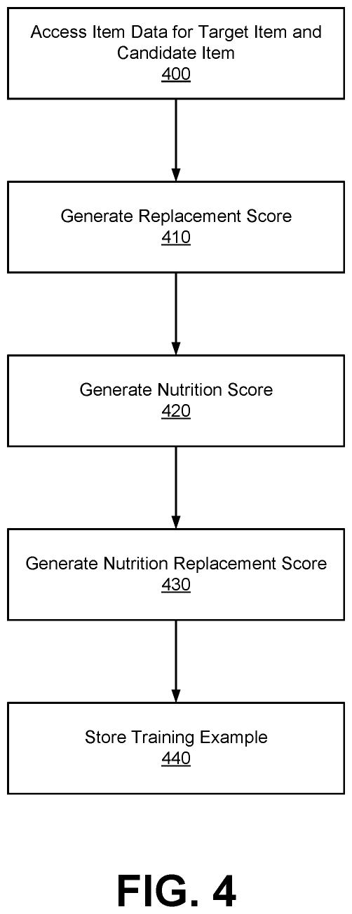

FIG. 4 is a flowchart for a method of generating training examples for a nutritional replacement model, in accordance with some embodiments. Alternative embodiments may include more, fewer, or different steps from those illustrated in FIG. 4, and the steps may be performed in a different order from that illustrated in FIG. 4. These steps may be performed by an online concierge system (e.g., online concierge system 140). Additionally, each of these steps may be performed automatically by the online concierge system without human intervention.

The online concierge system accesses 400 item data for a target item and item data for a candidate item. The online concierge system generates 410 a replacement score based on the accessed item data and generates 420 a nutrition score based on the item data for the candidate item. The online concierge system generates 430 a nutrition replacement score based on the replacement score and the nutrition score and stores 440 a training example based on the item data and the nutrition replacement score. The training example may include the item data for the target item and the candidate item and a label based on the nutrition replacement score.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor comprises one or more processors or processing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine learning models in the performance of their described functionalities. A “machine learning model,” as used herein, comprises one or more machine learning models that perform the described functionality. Machine learning models may be stored on one or more computer-readable media with a set of weights. These weights are parameters used by the machine learning model to transform input data received by the model into output data. The weights may be generated through a training process, whereby the machine learning model is trained based on a set of training examples and labels associated with the training examples. The training process may include: applying the machine learning model to a training example, comparing an output of the machine learning model to the label associated with the training example, and updating weights associated for the machine learning model through a back-propagation process. The weights may be stored on one or more computer-readable media, and are used by a system when applying the machine learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present), A is false (or not present) and B is true (or present), and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A non-transitory computer readable storage medium storing parameters for a nutritional replacement model, wherein the nutritional replacement model is produced by a process comprising:
generating a set of training examples for the nutritional replacement model, wherein generating a training example in the set of training examples comprises:
accessing item data describing a target item and item data describing a candidate item;
generating a replacement score based on the item data for the target item and the candidate item, wherein the replacement score indicates a likelihood that a user would approve the candidate item as a replacement for the target item;
generating a nutrition score based on the item data for the candidate item, wherein the nutrition score represents a nutritional value of the candidate item;
generating nutritional replacement score based on the replacement score and the nutrition score; and
storing a training example for the nutritional replacement model that comprises the item data for the candidate item, the item data for the target item, and a label based on the nutritional replacement score;
initializing the nutritional replacement model;
training the nutritional replacement model by iteratively updating a set of parameters for the nutritional replacement model based on each training example of the generated set of training examples for the nutritional replacement model; and
storing a final set of parameters for the trained nutritional replacement model to the computer readable storage medium as the parameters for the nutritional replacement model.

2. The non-transitory computer readable storage medium of claim 1, wherein the process further comprises:
accessing the item data from a training example for a replacement model, wherein the replacement model is a machine-learning model trained to predict a likelihood that a user will approve a first item as a replacement for a second item; and
generating the replacement score by accessing a label of the training example for the replacement model, wherein the label indicates whether a subject user selected the first item as a replacement for the user.

3. The non-transitory computer readable storage medium of claim 2, wherein the process further comprises:
accessing user data describing the subject user from the training example for the replacement model; and
storing the user data in the training example for the nutritional replacement model.

4. The non-transitory computer readable storage medium of claim 1, wherein generating the replacement score comprises:
applying a replacement model to the accessed item data, the replacement model is a machine-learning model trained to predict a likelihood that a user will approve a first item as a replacement for a second item.

5. The non-transitory computer readable storage medium of claim 1, wherein generating the nutrition score comprises:
applying a nutrition scoring model to item data for the candidate item, wherein the nutrition scoring model is a machine-learning model trained to compute a nutrition score representing the nutritional value of an item.

6. The non-transitory computer readable storage medium of claim 5, wherein the nutrition scoring model is trained on a set of training examples for the nutrition scoring model, wherein each of the set of training examples for the nutrition scoring model comprises item data for an item and a label indicating a nutrition score for the item based on feedback from a nutritionist.

7. The non-transitory computer readable storage medium of claim 1, wherein generating the nutrition score comprises:
identifying a set of recipes as healthy recipes based on item data describing items in each of the set of recipes; and
comparing items in the each of the set of recipes with items in recipes that are not in the set of recipes identified as healthy recipes.

8. The non-transitory computer readable storage medium of claim 7, wherein identifying a recipe in the set of recipes as healthy comprises:
applying a natural language process or a large language model to a title or a description of the recipe.

9. The non-transitory computer readable storage medium of claim 1, wherein generating the nutrition score comprises:
generating the nutrition score based on item data for the target item such that the nutrition score represents a relative nutritional value of the candidate item to the target item.

10. The non-transitory computer readable storage medium of claim 1, wherein generating the nutritional replacement score comprises:
computing a linear combination or a product of the nutrition score and the replacement score.

11. A method for training a machine-learning model comprising, at a computing system comprising a processor and a computer-readable medium:
generating a set of training examples for a nutritional replacement model, wherein generating a training example in the set of training examples comprises:
accessing item data describing a target item and item data describing a candidate item;
generating a replacement score based on the item data for the target item and the candidate item, wherein the replacement score indicates a likelihood that a user would approve the candidate item as a replacement for the target item;
generating a nutrition score based on the item data for the candidate item, wherein the nutrition score represents a nutritional value of the candidate item;
generating nutritional replacement score based on the replacement score and the nutrition score; and
storing a training example for the nutritional replacement model that comprises the item data for the candidate item, the item data for the target item, and a label based on the nutritional replacement score;
initializing the nutritional replacement model;
training the nutritional replacement model by iteratively updating a set of parameters for the nutritional replacement model based on each training example of the generated set of training examples for the nutritional replacement model; and
storing a final set of parameters for the trained nutritional replacement model to a computer-readable medium as the parameters for the nutritional replacement model.

12. The method of claim 11, wherein the process further comprises:
accessing the item data from a training example for a replacement model, wherein the replacement model is a machine-learning model trained to predict a likelihood that a user will approve a first item as a replacement for a second item; and
generating the replacement score by accessing a label of the training example for the replacement model, wherein the label indicates whether a subject user selected the first item as a replacement for the user.

13. The method of claim 12, wherein the process further comprises:
accessing user data describing the subject user from the training example for the replacement model; and
storing the user data in the training example for the nutritional replacement model.

14. The method of claim 11, wherein generating the replacement score comprises:
applying a replacement model to the accessed item data, the replacement model is a machine-learning model trained to predict a likelihood that a user will approve a first item as a replacement for a second item.

15. The method of claim 11, wherein generating the nutrition score comprises:
applying a nutrition scoring model to item data for the candidate item, wherein the nutrition scoring model is a machine-learning model trained to compute a nutrition score representing the nutritional value of an item.

16. The method of claim 15, wherein the nutrition scoring model is trained on a set of training examples for the nutrition scoring model, wherein each of the set of training examples for the nutrition scoring model comprises item data for an item and a label indicating a nutrition score for the item based on feedback from a nutritionist.

17. The method of claim 11, wherein generating the nutrition score comprises:
identifying a set of recipes as healthy recipes based on item data describing items in each of the set of recipes; and
comparing items in the each of the set of recipes with items in recipes that are not in the set of recipes identified as healthy recipes.

18. The method of claim 17, wherein identifying a recipe in the set of recipes as healthy comprises:
applying a natural language process or a large language model to a title or a description of the recipe.

19. The method of claim 11, wherein generating the nutrition score comprises:
generating the nutrition score based on item data for the target item such that the nutrition score represents a relative nutritional value of the candidate item to the target item.

20. A non-transitory computer-readable medium storing instructions that, when executed by a processor, cause the processor to perform operations comprising:
generating a set of training examples for a nutritional replacement model, wherein generating a training example in the set of training examples comprises:
accessing item data describing a target item and item data describing a candidate item;
generating a replacement score based on the item data for the target item and the candidate item, wherein the replacement score indicates a likelihood that a user would approve the candidate item as a replacement for the target item;
generating a nutrition score based on the item data for the candidate item, wherein the nutrition score represents a nutritional value of the candidate item;
generating nutritional replacement score based on the replacement score and the nutrition score; and
storing a training example for the nutritional replacement model that comprises the item data for the candidate item, the item data for the target item, and a label based on the nutritional replacement score;
initializing the nutritional replacement model;
training the nutritional replacement model by iteratively updating a set of parameters for the nutritional replacement model based on each training example of the generated set of training examples for the nutritional replacement model; and
storing a final set of parameters for the trained nutritional replacement model to a computer-readable medium as the parameters for the nutritional replacement model.
